(19) United States
(12) Patent Application Publication
(10) Pub. No.: US 2024/0316291 A1
(43) Pub. Date: Sep. 26, 2024

(54) RESPIRATORY DROPLET DELIVERY DEVICE WITH FACE SEAL AND METHODS OF USE

(71) Applicant: PNEUMA RESPIRATORY, INC., Boone, NC (US)

(72) Inventors: Charles Eric Hunter, Boone, NC (US); John H. Hebrank, Boone, NC (US); Chengjie Li, Shenzhen (CN); Judson Sidney Clements, Boone, NC (US)

(73) Assignee: PNEUMA RESPRIRATORY, INC., Boone, NC (US)

(21) Appl. No.: 18/383,354

(22) Filed: Oct. 24, 2023

Related U.S. Application Data
(63) Continuation of application No. PCT/US22/26176, filed on Apr. 25, 2022.
(60) Provisional application No. 63/179,212, filed on Apr. 24, 2021.

(51) Int. Cl. A61M 15/00; A24F 7/00; A24F 40/05; A24F 40/10; A24F 40/485; A61M 11/00; A61M 15/06

(52) U.S. Cl. CPC A61M 15/0025; A24F 7/00; A24F 40/05; A24F 40/10; A24F 40/485; A61M 11/003; A61M 15/06; A61M 2205/3331

(57) ABSTRACT

A droplet delivery device including a face seal within a cover of the droplet delivery device to seal over an ejector mechanism when the cover couples to a mouthpiece.

### CROSS-REFERENCE TO RELATED APPLICATIONS

The present application is a continuation of PCT Application No. PCT/US2022/026176, filed Apr. 25, 2022, which claims the benefit of priority of U.S. Provisional Application No. 63/179,212, filed Apr. 24, 2021, each of which is incorporated herein by reference.

### FIELD OF THE INVENTION

This disclosure relates to respiratory droplet delivery devices, and more specifically to droplet delivery devices for the delivery of fluids to the respiratory system.

### BACKGROUND OF THE INVENTION

The use of droplet generating devices for the delivery of substances to the respiratory system is an area of large interest. A major challenge is providing a device that delivers an accurate, consistent, and verifiable amount of substance, with a droplet size that is suitable for successful delivery of substance to the targeted area of the respiratory system.

Currently, most inhaler type systems such as metered dose inhalers (MDI) and pressurized metered dose inhalers (p-MDI) or pneumatic and ultrasonic-driven devices generally produce droplets with high velocities and a wide range of droplet sizes including large droplet that have high momentum and kinetic energy. Droplets and aerosols with such high momentum do not reach the distal lung or lower pulmonary passageways, but rather are deposited in the mouth and throat. As a result, larger total drug doses are required to achieve the desired deposition in targeted respiratory areas. These large doses increase the probability of unwanted side effects.

Aerosol plumes generated from current droplet delivery systems, as a result of their high ejection velocities and the rapid expansion of the substance carrying propellant, may lead to localized cooling and subsequent condensation, deposition and crystallization of substance onto the device surfaces. Blockage of device surfaces by deposited substance residue is also problematic.

Accordingly, there is a need for a droplet delivery device that delivers droplets of a suitable size range, avoids surface fluid deposition and blockage of apertures, with an amount that is verifiable, and provides feedback regarding correct and consistent usage of the device to users.

### DESCRIPTION OF THE DRAWINGS

The patent or application file contains at least one drawing executed in color.

FIG. 1 illustrates an exploded view of a droplet delivery device in an embodiment of the invention.

FIG. 2 illustrates a front perspective from above of a droplet delivery device in an embodiment of the invention.

FIG. 3 illustrates a partial cutaway view of a face seal of a droplet delivery device in a separated position is an embodiment of the invention.

FIG. 4 illustrates a partial cutaway view of a cap fitted on a droplet delivery device in an embodiment of the invention.

FIG. 5 illustrates a partial cutaway view of locking a cap of a droplet delivery device in an embodiment of the invention.

FIG. 6 illustrates a partial cutaway and enlarged view of a lock stage of a droplet delivery device in an embodiment of the invention.

FIG. 7 illustrates a cross-sectional view of a face seal blocking air flow paths of a droplet delivery device in an embodiment of the invention.

FIG. 8 illustrates a perspective view corresponded to a cross-section view of a self-sealing stopper of a droplet delivery device in an embodiment of the invention.

FIGS. 9A-12C provide schematic illustrations of alternative cut openings and seal plates of self-sealing stopper configurations in embodiments of the invention.

### DETAILED DESCRIPTION

Effective and efficient delivery of substances to desired areas of the respiratory system using droplet delivery devices, and the synchronization of the administration of droplets with the inspiration/expiration cycle using such devices has always posed a problem. For instance, optimum deposition in alveolar airways generally requires droplets with aerodynamic diameters in the ranges of 1 to 6 μm, with droplets below about 4 μm shown to more effectively reach the alveolar region of the lungs and larger droplets above about 6 μm shown to typically deposited on the tongue or strike the throat and coat the bronchial passages. Smaller droplets, for example less than about 1 μm, penetrate more deeply into the lungs and have a tendency to be exhaled.

Certain aspects of the disclosure relate to a breath actuated platform for delivery of inhaled substances, described herein as a respiratory droplet delivery device. The device provides substantial improvements over current inhaled delivery systems by improving precision, reliability, and delivery to a user. In embodiments, the device of the disclosure includes fully integrated monitoring capabilities designed to enhance user experience and compliance.

In certain aspects, the disclosure relates to a respiratory droplet delivery device for administering fluids to the respiratory system of a user with precise droplet size. In certain embodiments, the device comprises a body housing, a mouthpiece, a fluid cartridge having at least one fluid reservoir, and an ejector mechanism comprising at least one piezoelectric actuator in vibrational communication with at least one aperture plate with a plurality of openings formed through its thickness for ejecting droplets. The device may further comprise at least one differential pressure sensor configured to activate the ejector mechanism upon sensing a pre-determined pressure change within the device to thereby generate the ejected stream of droplets. In certain embodiments, the pressure sensor may be located in the mouthpiece, on the airflow side of the ejector mechanism.

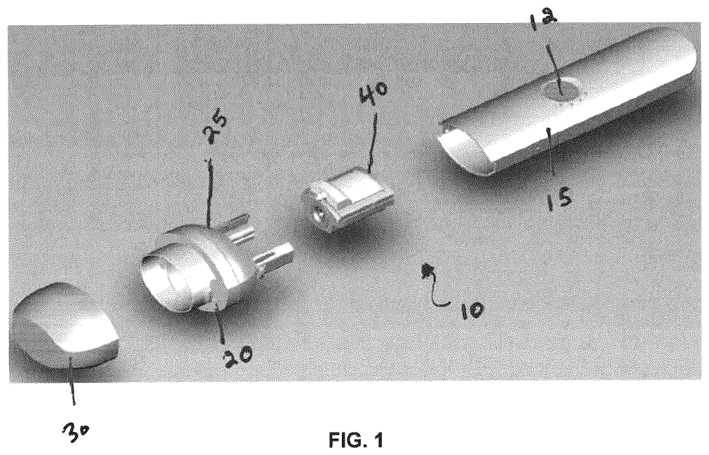

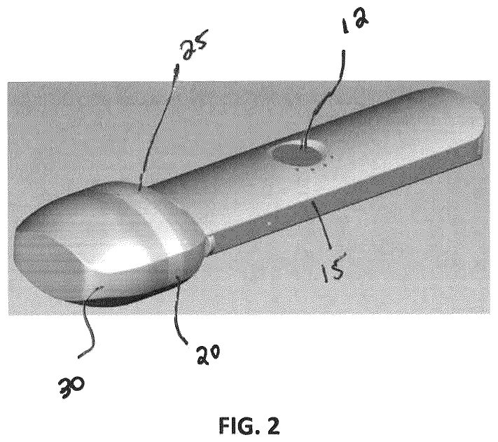

An exemplary droplet delivery device 10 of the disclosure is illustrated in FIGS. 1 and 2 with the cap/cover 30, mouthpiece 20, a combination ejector mechanism 25 and fluid cartridge 40, and a body housing 15 having a power activation button 12. As shown in FIG. 2, in certain aspects, the body housing 15 and mouthpiece 20 may fit together to enclose the fluid cartridge 50 and the ejector mechanism 25 within an enclosed device 10, and the cover 30 may interface with the mouthpiece 20 to close the mouthpiece.

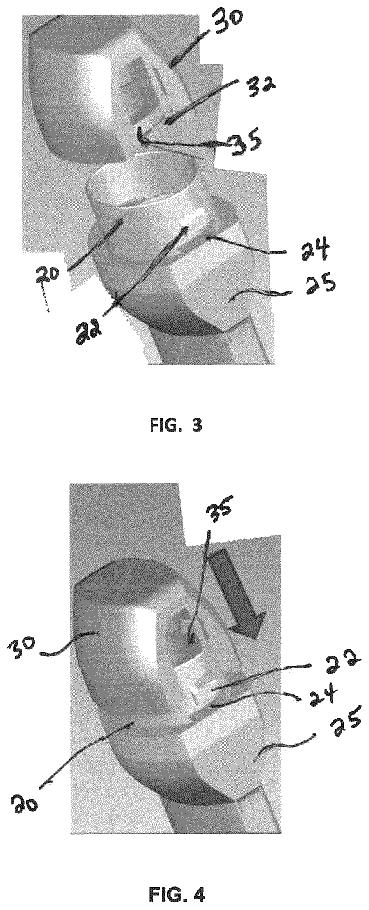

In certain embodiments, the body housing 15 may comprise batteries and electronics for controlling operation and actuation of the ejector mechanism 25, sensors, etc. The mouthpiece is generally located at an airflow exit of the device 10, and one or more airflow entrance ports 24 are generally located on the mouthpiece (as illustrated in FIG. 3) or body housing (not shown). The ejector mechanism 25 is located within the device so as to be in fluid communication with the fluid cartridge 40, so as to receive fluid from the fluid reservoir. The piezoelectric actuator is interfaced with the aperture plate 72 and operable to oscillate the aperture plate 72 at a frequency to thereby generate an ejected stream of droplets.

The fluid cartridge 40 may be removable from the device and replaceable. The fluid reservoir(s) may have a single or multiple administrations of substance to be delivered to a user. The ejector mechanism 25 may be interfaced with or located within the mouthpiece 20 or the fluid cartridge 40.

The ejector mechanism 25 may be orientated at various angles within the device, with respect to the direction of droplet generation, airflow through the device, and internal surface within the device. Without intending to be limited by theory, it is believed that orientation of the ejector mechanism 25 with respect to the direction of droplet generation, airflow through the device, and internal surfaces within the device serves to optimize droplet size distribution via inertial filtering, which filters and excludes larger droplets from the droplet plume.

For instance, in some embodiments, the ejector mechanism 25 may be oriented perpendicularly to the direction of airflow through the device, such that droplets are initially ejected into the direction of airflow. Such a configuration minimizes inertial filtering of generated droplets, allowing most droplets to flow in the entrained airflow within the mouthpiece (other than impacts of droplets at the sidewalls of the mouthpiece and inertial settling along the flowpath). In other embodiments, the ejector mechanism 25 may be orientated at an angle with respect to the direction of airflow through the device. By way of example, the ejector mechanism may be oriented at about 5° from perpendicular, about 10° from perpendicular, about 15° from perpendicular, about 20° from perpendicular, about 25° from perpendicular, about 30° from perpendicular, about 35° from perpendicular, about 40° from perpendicular, about 45° from perpendicular, etc. In such embodiments, the droplets may be ejected into the airflow at an angle, such that smaller droplets are able to flow in the entrained airflow within the mouthpiece 20, and larger droplets are more likely to impact the sidewalls of the mouthpiece 20 along the flowpath (or settle out along the flowpath).

The droplet delivery device may also have one or more sealing mechanisms, e.g., to protect the ejector mechanism and/or to minimize evaporation of fluid within the device. For example, in one embodiment, the device may include a sealing mechanism including a face seal configured to cover at least a portion of the aperture plate when not in use. Any suitable face seal may be used, for instance, a face seal may be part of a mouthpiece cap that is closed by the user after an inhalation. In certain embodiments, the cap may include an O-ring sealed, a spring loaded or biased face seal that presses against a metal (e.g., stainless steel, aluminum, or other alloy) surface within the mouthpiece but outside the aperture plate.

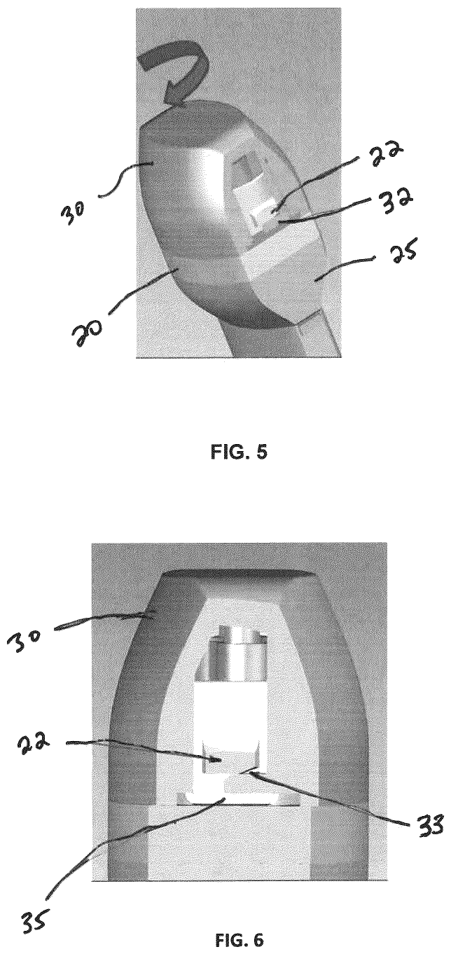

An exemplary cap face seal 35 and cap 30 coupling to mouthpiece 20 is shown in FIGS. 3-6. FIG. 3 illustrates the cap 30 and face seal 35 in a position separated from the rest of the droplet delivery device 10. The cap 30 includes at least one locking slider 32 that is configured to interface with at least one lock stage 22 positioned on the mouthpiece 20 of the device 10.

As shown in FIG. 4, the locking slider 32 inside the cap 30 contacts the lock stage 22 on the side of the mouthpiece 20 when the cap 30 is fitted onto the mouthpiece. One mating pair of locking slider/lock stage is illustrated in the figures, with another pair being located on the opposite side of the cap and mouthpiece (not shown). However, other configurations and orientations are envisioned as within the scope of the disclosure.

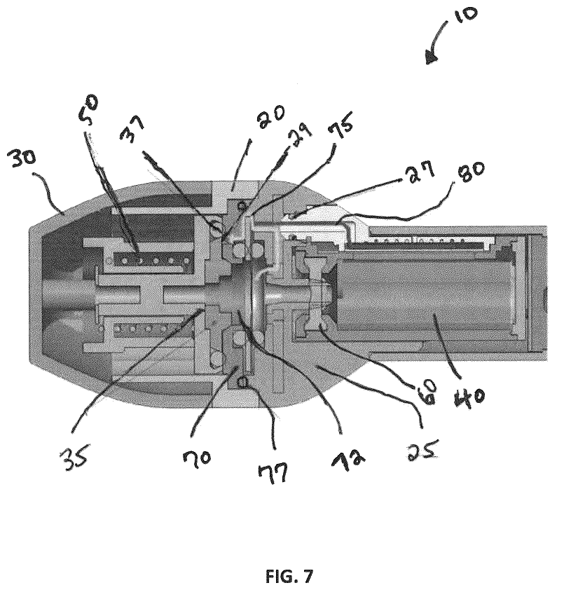

The face seal 35, including any optional O-ring, biasing member, spring-loading member (such as spring mechanism 50 shown in FIG. 7), and the like, can contact the aperture plate 72 of the ejector mechanism 25 to seal the aperture plate 72. With reference to 5, the cap may then be turned to lock the locking slider 32 in place under the inclined surface of the lock stage 22, thereby creating a force to compress the face seal 35 and create the sealing mechanism.

FIG. 6 illustrates an enlarged view of the lock stage 22, showing the inclined surface 33 under the lock stage 22 which creates a normal force to compress the face seal to create the sealing mechanism.

With reference to FIG. 7, a cross-section of an embodiment of the disclosure is illustrated, with the face seal 35 in place to block the air flow paths of the device, and thereby inhibit evaporation of fluid from the fluid cartridge reservoir 40. FIG. 7 further illustrates face seal O-ring 37 that resists sliding and rotating of the face seal 35. When cover 30 is closed, the face seal 35 seals the aperture plate and cartridge vent 29 to block air way 75 from the space behind the ejector mechanism 25 to vent 29. Face plate 70 may be coupled to an O-Ring 77 that seals the interface between the ejector aperture plate 72 and the cartridge seat. Air way 80 from the fluid cartridge 40 to the face plate vent is sealed by O-Ring 27 at the vent tube of fluid cartridge 40. In embodiments the face seal 35 and/or face plate 70 may comprise, among other materials, aluminum alloy or SU5316L steel. In embodiments, fluid cartridge 40 may comprise plastic, including Cyclic olefin copolymer (COC).

In another embodiment, the device 10 may include a sealing mechanism 60 at the interface of the fluid cartridge 40 and the ejector mechanism 25 to minimize evaporation of the fluid within the reservoir. The sealing mechanism 60 may include a sealing surface which covers any fluid exit paths when not in use and/or which covers the aperture plate when not in use. In yet other embodiments, the device may include a sealing mechanism to minimize evaporation at the connection point between the fluid cartridge and body housing. In some embodiments, the fluid cartridge may have a removable sealing tape which prevents evaporation prior to insertion or attachment to the device.

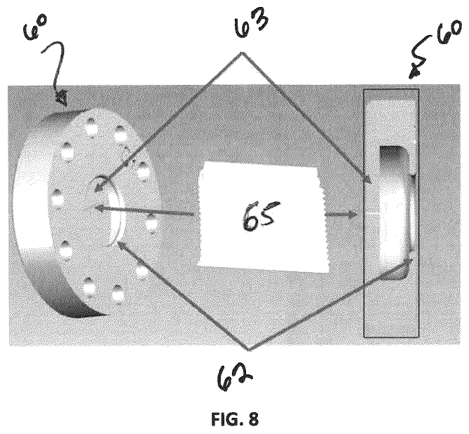

By way of non-limiting example, the sealing mechanism 60 at the interface between the fluid cartridge and the ejector mechanism may include a self-sealing polymer (e.g., rubber) type stopper. With reference to FIG. 8, an exemplary self-sealing stopper includes an opening cut 65 that allows fluid to flow from cartridge 40 to ejector mechanism 25 and aperture plate 75 when ejector mechanism 25 mates with fluid cartridge 40. Self-sealing stopper may also include a first seal ring 62 and second seal plate 63.

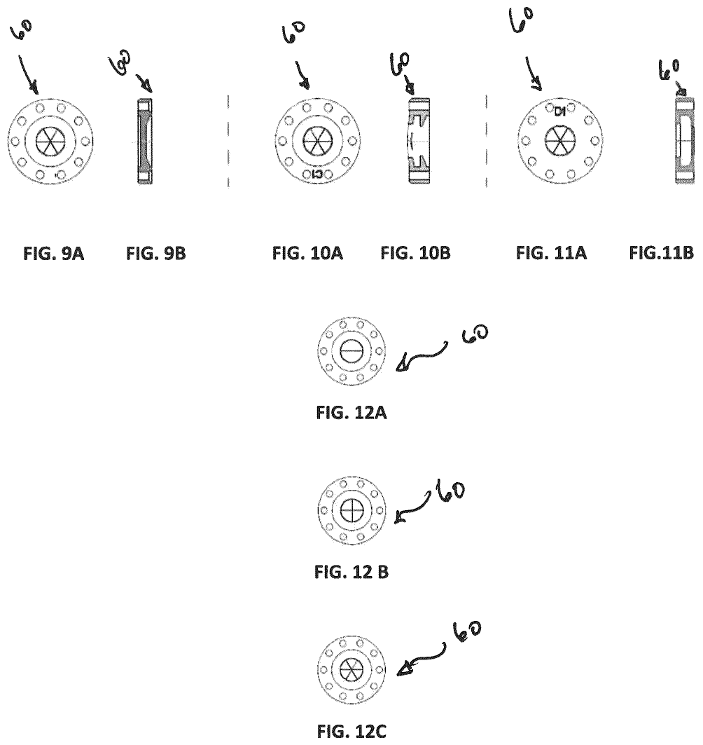

Alternative opening cut and seal plates of self-sealing stopper configurations are illustrated in FIGS. 9A-12C.

The self-sealing mechanism 60 may be located on the fluid cartridge 40 at the interface to the ejector mechanism 25. The mouthpiece 20 and/or the ejector mechanism 25 may include a fluid path (e.g., a protrusion or needle like extension) that may interface with or extend through the sealing mechanism 60 to create fluid communication between the fluid cartridge 40 and the ejector mechanism 25.

In certain embodiments, the fluid cartridge may further include fluid displacement elements (e.g., spheres or cylinders) within its volume that are formed from a material or include a material in its composition that has a density greater than that of the fluid to be dispensed. The fluid displacement elements are configured to move, roll, or otherwise be positioned within the fluid reservoir to the lowest point within the reservoir during use, thereby displacing fluid and reducing the dead space volume within the reservoir to improve fluid/aperture plate contact surface area.

In certain aspects, the present disclosure relates to the respiratory droplet delivery devices described herein for delivery a fluid as an ejected stream of droplets to the respiratory system of a user and related methods of delivering safe, suitable, and repeatable dosages to the respiratory system of a user. The present disclosure also includes the respiratory droplet delivery devices described herein capable of delivering a defined volume of fluid in the form of an ejected stream of droplets such that an adequate and repeatable high percentage of the droplets are delivered into the desired location within the airways, e.g., the alveolar airways of a user during use.

The present disclosure provides an respiratory droplet delivery device for delivery of a fluid as an ejected stream of droplets to the respiratory system of a user, the device comprising a housing, a mouthpiece, a fluid cartridge having at least one fluid reservoir for receiving a volume of fluid, an ejector mechanism including at least one respiratory actuator, and at least one aperture plate with an array of micron-sized openings for ejecting droplets, wherein the ejector mechanism is configured to vibrate the at least one aperture plate to eject a stream of droplets having an average ejected droplet diameter of less than about 6 microns, preferably less than about 5 microns, less than about 4 microns, less than about 3 microns, less than about 2.6 microns, less than about 2.3 microns, less than about 2 microns, less than about 1.6 microns, less than about 1.3 microns, less than about 1 micron, etc.

In specific embodiments, the ejector mechanism is electronically breath activated by at least one differential pressure sensor located within the housing of the respiratory droplet delivery device upon sensing a pre-determined pressure change within the mouthpiece. In certain embodiments, such a pre-determined pressure change may be sensed during an inspiration cycle by a user of the device, as will be explained in further detail herein.

In some aspects, the droplet delivery device further includes an air inlet flow element positioned in the airflow at the airflow entrance of the housing and configured to facilitate non-turbulent (i.e., laminar and/or transitional) airflow across the exit side of at least one aperture plate and to provide sufficient airflow to ensure that the ejected stream of droplets flows through the droplet delivery device during use. In some embodiments, the air inlet flow element may be positioned within the mouthpiece. In certain embodiments, the air inlet flow element may be positioned behind the exit side of the aperture plate along the direction of airflow, or in-line or in front of the exit side of the aperture plate along the direction of airflow.

In certain embodiments, the air inlet flow element comprises one or more openings formed there through and configured to increase or decrease internal pressure resistance within the droplet delivery device during use. For instance, the air inlet flow element comprises an array of one or openings. In the embodiments, the air inlet flow element comprises one or more baffles, e.g., wherein the one or more baffles comprise one or more airflow openings.

The airflow exit of the housing of the droplet delivery device through which the ejected aerosol of droplets exit as they are inhaled into a subject's airways, may be configured and have, without limitation, a cross sectional shape of a circle, oval, rectangular, hexagonal or other shape, while the shape of the length of the tube, again without limitation, may be straight, curved or have a Venturi-type shape.

In accordance with certain aspects of the disclosure, effective deposition into the lungs generally requires droplets less than about 5-6 μm in diameter, preferably less than about 5 μm, less than about 4 μm, less than about 3 μm, less than about 2.5 μm, less than about 2.3 μm, less than about 2 μm, less than about 1.6 μm, less than about 1.3 μm, less than about 1 μm, etc. Without intending to be limited by theory, to deliver fluid to the lungs a droplet delivery device must impart a momentum that is sufficiently high to permit ejection out of the device, but sufficiently low to prevent deposition on the tongue or in the back of the throat. Droplets below approximately this size are transported almost completely by motion of the airstream and entrained air that carry them and not by their own momentum.

In certain aspects, the present disclosure includes and provides an ejector mechanism configured to eject a stream of droplets within the respirable range of less than about 5-6 μm, preferably less than about 5 μm, less than about 4 microns, less than about 3 microns, less than about 2.5 microns, less than about 2.3 microns, less than about 2 microns, less than about 1.6 microns, less than about 1.3 microns, less than about 1 micron, etc. The ejector mechanism is comprised of a piezoelectric actuator vibrationally coupled to at least one aperture plate, as described herein. The aperture plate generally includes a plurality of openings formed through its thickness and the ejector mechanism oscillates the aperture plate (via its vibrational energy), which has fluid in contact with one surface of the aperture plate, to thereby generate a directed aerosol stream of droplets through the openings of the aperture plate into the lungs as the user inhales.

The ejected stream of droplets includes, without limitation, droplets formed from solutions, suspensions or emulsions which have viscosities in a range capable of droplet formation using the ejector mechanism and aperture plate. In certain aspects, the therapeutic agents may be delivered at a high dose concentration and efficacy, as compared to alternative dosing routes and standard inhalation technologies.

In certain embodiments, the droplet delivery devices of the disclosure may be used to deliver any suitable substance or agent to the respiratory system of a user. For example, the droplet delivery devices may be used to delivery therapeutic agents including small and large molecules. In certain embodiments, the respiratory droplet delivery devices of the disclosure may be used to treat various diseases, disorders and conditions by delivering therapeutic agents to the respiratory system of a subject. In this regard, the respiratory droplet delivery devices may be used to deliver therapeutic agents both locally to the respiratory system, and systemically to the body.

In certain embodiments, the devices and methods may be used to deliver a composition comprising an agent that may isolated or derived from cannabis. For instance, the agent may be a natural or synthetic cannabinoid, e.g., THC (tetrahydrocannabinol), THCA (tetrahydrocannabinolic acid), CBD (cannabidiol), CBDA (cannabidiolic acid), CBN (cannabinol), CBG (cannabigerol), CBC (cannabichromene), CBL (cannabicyclol), CBV (cannabivarin), THCV (tetrahydrocannabivarin), CBDV (cannabidivarin), CBCV (cannabichromevarin), CBGV (cannabigerovarin), CBGM (cannabigerol monomethyl ether), CBE (cannabielsoin), CBT (cannabicitran), and various combinations thereof. In other embodiments, the agent may be a ligand that bind the cannabinoid receptor type 1 (CBI), the cannabinoid receptor type 2 (CB2), or combinations thereof.

In particular embodiments, the agent may comprise THC, CBD, or combinations thereof. By way of example, the agent may comprise 95% THC, 98% THC, 99% THC, 95% CBD, 98% CBD, 99% CBD, etc.

In other embodiments, the devices and methods of the disclosure may be used to deliver a solution of nicotine or a salt thereof, e.g., including the water-nicotine azeotrope.

By way of non-limiting example, the nicotine or salt thereof may be the naturally occurring alkaloid compound having the chemical name S-3-(1-methyl-2-pyrrolidinyl)pyridine, which may be isolated and purified from nature or synthetically produced in any manner, or any of its occurring salts containing pharmacologically acceptable anions, such as hydrochloride, hydrobromide, hydroiodide, nitrate, sulfate or bisulfate, phosphate or acid phosphate, acetate, lactate, citrate or acid citrate, tartrate or bitartrate, succinate, maleate, fumarate, gluconate, pyruvate, saccharate, benzoate, methanesulfonate, ethanesulfonate, benzenesulfonate, p-toluene sulfonate, camphorate and pamoate salts. In other embodiments, the composition may further include any pharmacologically acceptable derivative, metabolite or analog of nicotine which exhibits pharmacotherapeutic properties similar to nicotine. Such derivatives and metabolites are known in the art, and include cotinine, norcotinine, nornicotine, nicotine N-oxide, cotinine N-oxide, 3-hydroxycotinine and 5-hydroxycotinine or pharmaceutically acceptable salts thereof.

In certain embodiments, the methods and droplet delivery devices of the disclosure may be used to treat various diseases, disorders and conditions by delivering agents to the respiratory system of a subject. In this regard, the droplet delivery devices may be used to deliver therapeutic agents both locally to the respiratory system, and systemically to the body. In certain embodiments, the methods and droplet delivery devices of the disclosure may be used to treat epilepsy, seizure disorders, pain, chronic pain, neuropathic pain, headache, migraine, arthritis, multiple sclerosis, anorexia, nausea, vomiting, anorexia, loss of appetite, anxiety, insomnia, etc. In other embodiments, the methods and in-line droplet delivery devices of the disclosure may be used to treat asthma and/or COPD.

In certain embodiments, the respiratory drug delivery device of the disclosure may be used to deliver scheduled and controlled substances such as narcotics for the highly controlled dispense of pain medications where dosing is monitored or otherwise controlled.

In certain embodiments, by way of non-limiting example, activation and/or droplet delivery may only enabled by a specific user identification by the device or via communication to the device, a doctor or pharmacy communication to the device, only in a specific location (such as the patient's residence, not near a school or other prohibited location, etc., as verified by GPS location on the user's smart phone), and/or it may be controlled by monitoring compliance with administration schedules, amounts, abuse compliances, etc. In certain aspects, this mechanism of highly controlled dispensing of substances can prevent the abuse or overdose of controlled substances.

In other embodiments, the respiratory droplet delivery device may be used to deliver therapeutic agents as an ejected stream of droplets to the respiratory system of a subject for the treatment or prevention of respiratory diseases or disorders such as asthma, chronic obstructive respiratory diseases (COPD) cystic fibrosis (CF), tuberculosis, chronic bronchitis, or pneumonia. In certain embodiments, the respiratory droplet delivery device may be used to deliver therapeutic agents such as COPD medications, asthma medications, or antibiotics. By way of non-limiting example, such therapeutic agents include albuterol sulfate, ipratropium bromide, tobramycin, fluticasone propionate, fluticasone furoate, tiotropium, glycopyrrolate, olodaterol, salmeterol, umeclidiniurn, and combinations thereof.

In other embodiments, the respiratory droplet delivery device may be used for the systemic delivery of therapeutic agents including small molecules, therapeutic peptides, proteins, antibodies, and other bioengineered molecules via the respiratory system. By way of non-limiting example, the in-line droplet delivery device may be used to systemically deliver therapeutic agents for the treatment or prevention of indications inducing, e.g., diabetesmellitus, rheumatoid arthritis, plaque psoriasis, Crohn's disease, hormone replacement, neutropenia, nausea, influenza, etc.

By way of non-limiting example, therapeutic peptides, proteins, antibodies, and other bioengineered molecules include: growth factors, insulin, vaccines (Prevnor Pneumonia, Gardasil—HPV), antibodies (Keytruda (pembrolizumab), Opdivo (nivolumab), Avastin (bevacizumab), Humira (adalimumab), Remicade (infliximab), Herceptin (trastuzumab)), Fc Fusion Proteins (Enbrel (etanercept), Orencia (abatacept)), hormones (Elonva-long acting FSH, Growth Hormone), enzymes (Pulmozyme-rHu-DNAase-), other proteins (Clotting factors, Interleukins, Albumin), gene therapy and RNAi, cell therapy (Provenge-Prostate cancer vaccine), antibody drug conjugates-Adcetris (Brentuximab vedotin for HL), cytokines, anti-infective agents, polynucleotides, oligonucleotides (e.g., gene vectors), or any combination thereof; or solid droplets or suspensions such as Flonase (fluticasone propionate) or Advair (fluticasone propionate and salmeterol xinafoate).

As discussed above, effective delivery of droplets deep into the lung airways require droplets that are less than about 5-6 microns in diameter, specifically droplets with mass mean aerodynamic diameters (MMAD) that are less than about 5 microns. The mass mean aerodynamic diameter is defined as the diameter at which 50% of the droplets by mass are larger and 50% are smaller. In certain aspects of the disclosure, in order to deposit in the alveolar airways, droplets in this size range must have momentum that is sufficiently high to permit ejection out of the device, but sufficiently low to overcome deposition onto the tongue (soft palate) or pharynx.

In certain aspects, the droplet delivery device is capable of delivering a defined volume of fluid in the form of an ejected stream of droplets having a small diameter such that an adequate and repeatable high percentage of the droplets are delivered into the desired location within the airways, e.g., the alveolar airways of the subject during use. In certain embodiments, the droplet diameters may range from about 0.7 μm to about 5 μm, about 0.7 μm to about 4.7 μm, about 0.7 μm to about 4 μm, about 0.7 μm to about 2.5 μm, about 0.7 μm to about 1.3 μm, etc.

In other aspects of the disclosure, methods for generating an ejected stream of droplets for delivery to the respiratory system of user using the droplet delivery devices of the disclosure are provided. In certain embodiments, the ejected stream of droplets is generated in a controllable and defined droplet size range. By way of example, the droplet size range includes at least about 50%, at least about 60%, at least about 70%, at least about 85%, at least about 90%, between about 50% and about 90%, between about 60% and about 90%, between about 70% and about 90%, between about 70% and about 95%, etc., of the ejected droplets are in a respirable range of below about 6 μm, preferably below about 5 μm.

In other embodiments, the ejected stream of droplets may have one or more diameters, such that droplets having multiple diameters are generated so as to target multiple regions in the airways (mouth, tongue, throat, upper airways, lower airways, deep lung, etc. By way of example, droplet diameters may range from about 0.25 μm to about 200 μm, about 0.25 μm to about 100 μm, about 0.25 μm to about 60 μm, about 0.25 μm to about 40 μm, about 0.25 μm to about 20 μm, about 0.25 μm to about 5 μm, about 0.25 μm to about 4.7 μm, about 0.25 μm to about 4 μm, about 6 μm to about 50 μm, trout 10 μm to 100 μm, about 10 μm to 50 μm, about 10 μm to 40 μm, about 10 μm to 30 μm, about 10 μm to 20 μm, about 5 μm to about 10 μm, about 0.7 μm to about 5 μm, about 0.7 un to about 4.7 μm, about 0.7 μm to about 4 μm, about 0.7 μm to about 2.5 μm, about 0.7 μm to about 1.3 μm, and combinations thereof.

In particular embodiments, at least a fraction of the droplets have diameters in the respirable range, while other droplets may have diameters in other sizes so as to target non-respirable locations (e.g., larger than about 5 μm). Illustrative ejected droplet streams in this regard might have 50%-70% of droplets in the respirable range (less than about 5 μm), and 30%-50% outside of the respirable range, e.g., so as to target the mouth and/or throat (about 5 μm-about 10 μm, about 5 μm-about 20 μm, about 5 μm-about 30 μm, about 10 μm-about 30 μm, etc.)

In certain configurations, a single piezoelectric actuator and single aperture plate may be used to eject a stream of droplets having droplets with more than one diameter. In other embodiments, multiple piezoelectric actuators and multiple aperture plates may be used (together with multiple fluid cartridges or a single fluid cartridge with multiple fluid reservoirs interfaced with multiple aperture plates). In yet other embodiments, a single piezoelectric actuator with multiple aperture plates, again together with multiple fluid cartridges or a single fluid cartridge with multiple fluid reservoirs, may be used. By way of non-limiting example, a stream of droplets having some droplets with an average droplet diameter of about 0.25 μm to about 5, about 0.7 pinto about 5 μm, about 0.7 μm to about 4.7 μm, about 0.7 μm to about 4 μm, about 0.7 μm to about 2.5 μm, about 0.7 μm to about 1.3 μm, etc., and other droplets having an average droplet diameter of about 10 μm to 100 μm, about 10 μm to 50 μm, about 10 μm to 40 μm, about 10 μm it 30 μm, about 10 μm to 20 μm, etc., may be ejected. In some embodiments, the smaller droplets may be a substance for delivery to the lungs, e.g., nicotine, while the larger droplets may be a substance for delivery to the mouth and throat, e.g., a flavorant. In other embodiments, the substance to be delivered via the smaller and larger droplets may be the same. For instance, in some embodiments, the substance may be the same, but may be delivered to the lungs via the smaller droplets at one concentration (dosage), and delivered to the mouth and/or throat via the larger droplets at a second concentration. In some embodiments, the substance may be nicotine, a cannabinoid, or a medicament.

In another aspect of the disclosure, methods for delivering safe, suitable, and repeatable dosages of a substance, e.g., a medicament, to the respiratory system using the droplet delivery devices of the disclosure are provided. The methods deliver an ejected stream of droplets to the desired location within the respiratory system of the subject, including the deep lungs and alveolar airways.

In certain aspects of the disclosure, a respiratory droplet delivery device for delivery an ejected stream of droplets to the respiratory system of a user is provided. The respiratory droplet delivery device generally includes a housing, a mouthpiece positioned at the airflow exit side of the housing, a fluid cartridge disposed in or in fluid communication with the housing including a fluid reservoir for receiving a volume of fluid, an ejector mechanism including a piezoelectric actuator in vibrational communication with an aperture plate for ejecting a stream of droplets, and at least one differential pressure sensor positioned within the housing. The differential pressure sensor is configured to electronically breath activate the ejector mechanism upon sensing a pre-determined pressure change within the mouthpiece, and the ejector mechanism is configured oscillate the aperture plate to thereby generate an ejected stream of droplets.

In certain embodiments, the mouthpiece may be interfaced with (and optionally removable and/or replaceable), integrated into, or part of the housing. In other embodiments, the mouthpiece may be interfaced with (and optionally removable and/or replaceable), integrated into, or part of the fluid cartridge.

In certain embodiments, the respiratory droplet delivery device is comprised of a separate fluid cartridge including a fluid reservoir and aperture plate, and a handheld base unit (e.g., housing/body) including an ejector mechanism, a differential pressure sensor, a microprocessor and three AAA batteries. In certain embodiments, the handheld base unit also includes a mouthpiece, optionally removable, an optional mouthpiece cover, and an optional ejector plate seal. The microprocessor r controls dose delivery, dose counting and software designed monitoring parameters that can be transmitted through blue-tooth technology. The ejector mechanism optimizes droplet delivery to the lungs by creating an ejected droplet stream in cooperation with the aperture plate in a predefined range with a high degree of accuracy and repeatability. Initial droplet studies show at least 65% to 70% of droplets ejected from the device are in the respirable range (e.g., 1-5 μm).

In certain embodiments, the respiratory droplet delivery device may include a fluid cartridge that may be replaceable or disposable either on a periodic basis, e.g., a daily, weekly, monthly, as-needed, etc. basis, as may be suitable for a prescription or over-the-counter medication. The fluid reservoir may be prefilled and stored in a pharmacy for dispensing to patients or filled at the pharmacy or elsewhere by using a suitable injection means such as a hollow injection syringe driven manually or driven by a micro-pump. The syringe may fill the reservoir by pumping fluid into or out of a rigid container or other collapsible or non-collapsible reservoir. In certain aspects, such a fluid cartridge may minimize and prevent buildup of surface deposits or surface microbial contamination on the aperture plate, owing to its short in-use time.

In certain aspects of the disclosure, the ejector mechanism, fluid cartridge, and housing/mouthpiece function to generate a plume with droplet diameters less than about 5 μm.

As discussed above, in certain embodiments, the ejector mechanism is powered by electronics in the device housing, and the fluid reservoir may carry sufficient substance for a single dose, a few doses, or several hundred doses of medicament.

In certain aspects, the devices of the disclosure eliminate the need for user/device coordination by using a differential pressure sensor to initiate the piezoelectric ejector in response to the onset of inhalation.

As described herein, in certain embodiments, the respiratory droplet delivery device may be turned on and activated for use by inserting the fluid cartridge into the base unit, opening the mouthpiece cover, and/or switching an on/off switch/slide bar. In certain embodiments, visual and/or audio indicators may be used to indicate the status of the device in this regard, e.g., on, off, stand-by, preparing, etc. By way of example, one or more LED lights may turn green and/or flash green to indicate the device is ready for use. In other embodiments, visual and/or audio indicators may be used to indicate the status of the fluid cartridge, including the number of inhalations taken, the number of inhalations remaining, instructions for use, etc.

For example, and LED visual screen may indicate a inhalation counter numerical display with the number of remaining inhalations in the reservoir.

As described in further detail herein, during use as a user inhales through the mouthpiece of the housing of an respiratory droplet delivery device of the disclosure, a differential pressure sensor within the housing detects inspiratory flow, e.g., by measuring the pressure drop across a Venturi plate or other suitable pressure sensor at the back of the mouthpiece. When a threshold pressure decline (e.g., 8 Om) is attained, the microprocessor activates the ejector mechanism, which in turn generates an ejected stream of droplets into the airflow of the device that the user inhales through the mouthpiece. In certain embodiments, audio and/or visual indicates may be used to indicate that dosing has been initiated, e.g., one or more LEDs may illuminate green. The microprocessor then deactivates the ejector at a designated time after initiation so as to achieve a desired administration dosage, e.g., 1-1.45 seconds. Alternatively, the microprocessor may deactivate when the pressure sensor indicates that inhalation is no longer detected. In such embodiments, thresholds may be set to ensure that overdose and abuse does not occur.

In certain embodiments, as described in further detail herein, the device may provide visual and/or audio indicators to facilitate proper dosing, e.g., the device may emit a positive chime sound after the initiation of dosing, indicating to the user to begin holding their breath for a designated period of time, e.g., 10 seconds. During the breath hold period, e.g., the three green LEDs may blink. Additionally, there may be voice co-instructing the patient on proper times to exhale, inhale and hold their breath, with an audio indicator of a breath hold countdown.

Following dosing, the respiratory droplet delivery device may turned off and deactivated in any suitable manner, e.g., by closing the mouthpiece cover, switching an on/off switch/slide bar, timing out from non-use, removing the drug ampoule, etc. If desired, audio and/or visual indicators may prompt a user to deactivate the device, e.g., by flashing one or more red LED lights, providing voice commands to close the mouthpiece cover, etc.

In certain embodiments, the respiratory droplet delivery device may include an ejector mechanism closure system that seals the aperture plate when not in use to protect the integrity of the aperture plate and to minimize and prevent contamination and evaporation of the fluid within the reservoir. For example, in some embodiments, the device may include a mouthpiece cover that comprises a rubber plug that is sized and shaped to seal the exit side surface of the aperture plate when the cover is closed. In other embodiments, the mouthpiece cover may trigger a slide to seal the exit side surface of the aperture plate when the cover is closed. Other embodiments and configurations are also envisioned, e.g., manual slides, covers, and plugs, etc. In certain aspects, the microprocessor may be configured to detect when the ejector mechanism closure, aperture plate seal, etc. is in place, and may thereafter deactivate the device

Several features of the device allow precise dosing of specific droplet sizes. Droplet size is set by the diameter of the holes in the aperture plate which are formed with high accuracy. By way of example, the exit side holes in the aperture plate may range in size from 1 μm to 6 μm, from 2 μm to 5 μm, from 3 μm to 5 μm, from 3 μm to 4 μm, etc. In other embodiments, as described herein, if multiple sizes of droplets are desired, the aperture plate may be configured with areas of holes having multiple diameters. For example, the aperture plate may have concentric rings having hole diameters of differing sizes, an internal area having a first hole size diameter, and an external ring having a different hole size diameter, one side having a first size hole diameter and the other side having a second size hole diameter, etc. Ejection rate, in droplets per second, is generally fixed by the frequency of the aperture plate vibration, e.g., 108-kHz, which is actuated by the microprocessor. In certain embodiments, there is less than a 50-millisecond lag between the detection of the start of inhalation and full droplet generation.

Other aspects of the device of the disclosure that allow for precise dosing of specific droplet sizes include the production of droplets within the respirable range early in the inhalation cycle, thereby minimizing the amount of drug product being deposited in the mouth or upper airways at the end of an inhalation. In addition, the design of the fluid cartridge allows the aperture plate surface to be wetted and ready for ejection without user intervention, thus obviating the need for shaking and priming. Further, the design of the fluid cartridge together with the face seal limits fluid evaporation from the reservoir to less than 150 μL to 350 μL per month.

The device may be constructed with materials currently used in FDA cleared devices. Standard manufacturing methods may be employed to minimize extractables. Any suitable material may be used to form the housing of the droplet delivery device. In particular embodiment, the material should be selected such that it does not interact with the components of the device or the fluid to be ejected (e.g., drug or medicament components). For example, polymeric materials suitable for use in pharmaceutical applications may be used including, e.g., gamma radiation compatible polymer materials such as polystyrene, polysulfone, polyurethane, phenolics, polycarbonate, polyimides, aromatic polyesters (PET, PETG), etc.

The aperture plate maybe metallic or polymer with openings about the diameter of the desired droplets (as discussed further herein). By way of non-limiting example, the aperture plate may formed from silicon, silicon carbide, nickel palladium, or a high stiffness polymer such as polyether ether ketone (PEEK), poly-amide, Kapton or Ultra High Molecular Weight Polyethylene (UHMWPE). The aperture plate may have an array of opening ranging from, e.g., 100 to 10,000 openings, 500 to 10,000 openings, etc. The openings may generally have a diameter similar to that of the desired droplets, e.g., of 1 μm to 100 μm diameter, as described further herein. When using a polymer aperture plate, the holes may be produced by rolling, stamping, laser ablation, bulk etching or other known micro-machining processes. When using silicon and SiC aperture plates, the openings may be formed using typical semiconductor processes. In addition, the aperture plate area can be formed to have a domelike shape to increase the stiffness of the aperture plate and creating uniform ejection accelerations.

In certain embodiments, the aperture plate may include various coatings on one or more surfaces. For example, in certain embodiments, the aperture plate may include hydrophilic coating on one or more surfaces. In one embodiment, the aperture plate includes a hydrophilic coating on the fluid entrance side of the aperture plate (fluid reservoir facing side of the aperture plate), a hydrophilic coating within at least a portion of the interior of one or more openings, or combinations thereof. In other embodiments, the aperture plate may include a hydrophobic coating on the droplet exit or side of the aperture plate—alone in combination with one or more hydrophilic coatings. A gas or liquid process may be used to form the hydrophobic and hydrophilic surfaces. For example, hydrophilic and hydrophobic surfaces can be formed using liquid coating, sputtering, CVD, plasma deposition, ion implantation, etc.

In certain aspects, the respiratory droplet delivery device is capable of delivering a defined volume of fluid in the form of an ejected stream of droplets having a small diameter such that an adequate and repeatable high percentage of the droplets are delivered into the desired location within the airways, e.g., the alveolar airways of the subject during use. In certain embodiments, the droplet diameters may range from about 0.7 μm to about 5 μm, about 0.7 μm to about 4.7 μm, about 0.7 μm to about 4 μm, about 0.7 μm to about 2.5 μm, about 0.7 μm to about 1.3 μm, etc.

In certain embodiments, the disclosure provides a respiratory droplet delivery device for delivery of a fluid as an ejected stream of small droplets to the respiratory system of a subject, the device comprising a housing, a mouthpiece positioned at the airflow exit side of the housing, a fluid cartridge disposed in or in fluid communication with the housing including at least one fluid reservoir for receiving at least one volume of fluid, an ejector mechanism including a piezoelectric actuator in vibrational communication with an aperture plate having a desired surface contact angle on at least the fluid intake surface thereof, and at least one differential pressure sensor positioned within the housing, wherein the ejector mechanism is configured to eject a stream of droplets having an average ejected droplet diameter of less than about 4 microns, less than about 3 microns, less than about 2 microns, less than about 1.5 microns, less than about 1 microns, etc.

As discussed herein, in certain aspects, the respiratory droplet delivery device may include an ejector mechanism having a aperture plate wherein the surface is configured to facilitate generation of droplets with the desired droplet size distribution, e.g., less than 4 μm, less than about 3 microns, less than about 2 microns, less than about 1.5 microns, less than about 1 microns, etc.

In certain embodiments, to facilitate generation of droplets with the desired droplet size distribution, the surface of the aperture plate may be configured (e.g., treated, coated, surface modified, or a combination thereof) to provide a desired surface contact angle at the fluid intake surface of less than about 50 degrees, less than about 40 degrees, less than about 35 degrees, less than about 30 degrees, less than about 20 degrees, less than about 10 degrees, between about 10 and about 35 degrees, between about 15 and about 35 degrees, etc.

In certain embodiments, the aperture plate may be coated on at least the fluid intake side of the aperture plate with a hydrophilic polymer to achieve the desired surface contact angle. In other embodiments, the aperture plate may be coated on at least a portion of the interior surface of one or more openings, within the entire interior surface of one or more openings, on both the fluid intake surface and the fluid ejection surface of the aperture plate, and combinations thereof.

Without intending to be limited by theory, the hydrophilic coating is believed to more effectively attract an aqueous composition into the openings of the ejector aperture plate during the vibration of the aperture plate by the piezo element, thereby increasing the mass flow of aerosol droplets out of the aperture plate.

Any known hydrophilic polymer suitable for use in medical applications may be used. In certain aspects of the disclosure, hydrophilic surfaces may be created on metallic ejector aperture plates by increasing the surface energy through creation of a polar surface.

Exemplary methods to increase surface energy comprise forming an oxide surface on the metallic ejector aperture plate which is polar. The strength of the hydrophilic effect is measured by the angle between the edge of a droplet of water and the surface of the metal. A surface is considered to be hydrophilic when that angle is less than about 50 degrees, and considered to be super hydrophilic when that angle is less than about 10 to 20 degrees (droplet tends to spread out across the surface).

In accordance with aspects of the disclosure, exemplary methods for creating a hydrophilic surface on the fluid side of a metallic aperture plate including dip coating methods and chemical deposition methods. Dip coating methods comprise dipping the metal ejector aperture plate into a solution comprising a desired coating and a solvent, which solution will form a hydrophilic coating on the metal when the solvent evaporates. Chemical depositions methods include known deposition methods, e.g., plasma etch, plasma coating, plasma deposition, CVD, electroless plating, electroplating, etc., wherein the chemical deposition uses a plasma or vapor to open the bonds on the surface of the metal so that oxygen or hydroxyl molecules attach to the surface rendering it polar. In certain embodiments, the deposited hydrophilic layer is significantly thinner than the opening size such that it does not impact the size of the generated droplets.

Any suitable hydrophilic coating to achieve the desired surface contact angle on the fluid intake surface of the ejector aperture plate may be used. Exemplary hydrophilic coating materials include, but are not limited to siloxane based coatings, isocyante based coatings, ethylene oxide based coatings, polyisocyanate based coatings, hydrocyclosiloxane based coatings, hydroxyalkylmethacrylate based coatings, hydroxyalkylacrylate based coatings, glycidylmethacrylate based coatings, propylene oxide based coatings, N-vinyl-2-pyrrolidone based coatings, latex based coatings, polyvinylchloride based coatings, polyurethane based coatings, etc.

By way of non-limiting example, a suitable hydrophilic coating may comprise a single layer hydrophilic surface formed by a process of cleaning the intended surface with a low pressure plasma and then dipping the surface into a solution of organophosphorous acids which self-assemble into a polar monolayer (e.g., see Aculon U.S. Pat. No. 8,658,258A, which is incorporated herein by reference). These layers are typically less than 10 nm thick, which is significantly less than a micron-sized hole. Contact angles as low as 10 degrees can be achieved using such coatings.

In other embodiments, the aperture plate may optionally be coated on the fluid exit side with a hydrophobic coating. Any known hydrophobic polymer suitable for use in medical applications may be used, e.g., polytetrafluoroethylene (Teflon), siloxane based coatings, paraffin, polyisobutylene, etc. The surface of the hydrophobic coating may be chemically or structurally modified or treated to further enhance or control the surface contact angle, as desired. In certain embodiments, the aperture plate may be coated with a siloxane based coating to provide an initial hydrophobic coating, which siloxane based coating is thereafter masked or shielded in a suitable manner on the fluid exit side. Following masking, the masked aperture plate may thereafter be exposed to an oxidizing treatment to render the siloxane coating hydrophilic on the exposed (unmasked) portions thereof, i.e., the fluid intake surfaces. In this manner, in certain embodiments of the disclosure, the same siloxane-based coating may provide both hydrophilic and hydrophobic coatings to surfaces of the aperture plate. By way of example, such siloxane coatings may be selected from siloxanes known for use in medical applications, such as 2,4,6,8-Tetramethylcyclotetrasiloxane, or 1,1,3,3-Tetramethyldisiloxane.

As described above, in certain embodiments, aperture plates may be formed from silicon or silicon carbide. Without being limited, both of these materials can be formed by bulk micro-machining processes such as wet etching.

In certain aspects, the aperture plate may be bonded to the fluid cartridge after filling the cartridge. Further, if desired, the aperture plate may be bonded to an intermediary structural material like a stainless steel annulus to reduce costs by minimizing the ejector plate, or to increase the aperture plate stiffness or to facilitate attachment to the cartridge. With polymer materials, the aperture plate may have raised ribs at intervals to stiffen the aperture plate against flexure. Ribs can be produced by rolling or stamping in a polymer heated above its transition temperature. The fluid reservoir maybe constructed of any suitable materials for the intended pharmaceutical use. In particular, the agent contacting portions may be made from material compatible with the desired agent(s), e.g., nicotine, albuterol sulfate and ipratropium bromide.

By way of example, in certain embodiments, the agent only contacts the inner side of the drug reservoir and the inner face of the aperture plate. In certain embodiments, the fluid reservoir may be configured to hold a single dose or multiple doses of agent. By way of example, the fluid reservoir may hold between 10 to 2000 μL of fluid.

In certain embodiments, the device mouthpiece may be removable, replaceable and maybe cleaned. Similarly, the device housing and fluid cartridge can be cleaned by wiping with a moist cloth. In certain embodiments, the mouthpiece may be interfaced with (and optionally removable and/or replaceable), integrated into, or part of the housing. In other embodiments, the mouthpiece may be interfaced with (and optionally removable and/or replaceable), integrated into, or part of the fluid cartridge.

Again, any suitable material may be used to form the mouthpiece of the droplet delivery device. In particular embodiment, the material should be selected such that it does not negatively interact with the components of the device or the fluid to be ejected (e.g., drug or medicament components). For example, polymeric materials suitable for use in pharmaceutical applications may be used including, e.g., gamma radiation compatible polymer materials such as polystyrene, polysulfone, polyurethane, phenolics, polycarbonate, polyimides, aromatic polyesters (PET, PETG), etc. In certain embodiments, the mouthpiece may be removable, replaceable and sterilizable. This feature improves sanitation for drug delivery by providing a mechanism to minimize buildup of aerosolized medication within the mouthpiece and by providing for ease of replacement, disinfection and washing. In one embodiment, the mouthpiece tube may be formed from sterilizable and transparent polymer compositions such as polycarbonate, polyethylene or polypropylene, as discussed herein.

In certain aspects of the disclosure, an electrostatic coating may be applied to the one or more portions of the housing, e.g., inner surfaces of the housing along the airflow pathway such as the mouthpiece, to aid in reducing deposition of ejected droplets during use due to electrostatic charge build-up. Alternatively, one or more portions of the housing may be formed from a charge-dissipative polymer. For instance, conductive fillers are commercially available and may be compounded into the more common polymers used in medical applications, for example, PEEK, polycarbonate, polyolefins (polypropylene or polyethylene), or styrenes such as polystyrene or acrylic-butadiene-styrene (ABS) copolymers. Alternatively, in certain embodiments, one or more portions of the housing, e.g., inner surfaces of the housing along the airflow pathway such as the mouthpiece, may be coated with anti-microbial coatings, or may be coated with hydrophobic coatings to aid in reducing deposition of ejected droplets during use. Any suitable coatings known for such purposes may be used, e.g., polytetrafluoroethylene (Teflon).

Any suitable differential pressure sensor with adequate sensitivity to measure pressure changes obtained during standard inhalation cycles may be used, e.g., ±5 SLM, 10 SLM, 20 SLM, etc. For instance, pressure sensors from Sensirion, Inc., SDP31 or SDP32 (U.S. Pat. No. 7,490,511 B2) are particularly well suited for these applications.

In certain aspects, the microprocessor in the device may be programmed to ensure exact timing and actuation of the ejector mechanism in accordance with desired parameters, e.g., based duration of piezoelectric activation to achieve desired dosages, etc. In certain embodiments, the device includes or interfaces with a memory (on the device, smartphone, App, computer, etc.) to record the date-time of each ejection event, as well as the user's inhalation flow rate during the dose inhalation to facilitate user monitoring, as well as drug ampoule usage monitoring. For instance, the microprocessor and memory can monitor doses administered and doses remaining in a particular drug ampoule. In certain embodiments, the drug ampoule may comprise components that include identifiable information, and the base unit may comprise components that may “read” the identifiable information to sense when a drug ampoule has been inserted into the base unit, e.g., based on a unique electrical resistance of each individual ampoule, an RFID chip, or other readable microchip (e.g., cryptoauthentication microchip). Dose counting and lockouts may also be preprogramed into the microprocessor.

In certain embodiments of the present disclosure, the signal generated by the pressure sensors provides a trigger for activation and actuation of the ejector mechanism to thereby generate droplets and delivery droplets at or during a peak period of a patient's inhalation (inspiratory) cycle and assures optimum deposition of the plume of droplets and delivery of the medication into the respiratory airways of the user. In accordance with certain aspects of the disclosure, the respiratory droplet delivery device provides a reliable monitoring system that can date and time stamp actual delivery of substance, and record/store inspiratory airflow in a memory (on the device, smartphone, App, computer, etc.). Blue tooth or other wireless communication capabilities may then permit the wireless transmission of the data.

Bluetooth communication in the device will communicate date, time and number of actuations per session to the user's smartphone. Software programing can provide charts, graphics, medication reminders and warnings to patients and whoever is granted permission to the data. The software application will be able to incorporate multiple uses and users of the device (e.g. multiple substances, different users, etc.). The device of the present disclosure is configured to dispense droplets during the correct part of the inhalation cycle, and can including instruction and/or coaching features to assist patients with proper device use, e.g., by instructing the holding of breath for the correct amount of time after inhalation. The device of the disclosure allows this dual functionality because it may both monitor air flow during the inhalation, and has internal sensors/controls which may detect the end of inhalation (based upon measured flow rate) and can cue the patient to hold their breath for a fixed duration after the inhalation ceases.

In one exemplary embodiment, a patient may be coached to hold their breath with an LED that is turned on at the end of inhalation and turned off after a defined period of time (i.e., desired time period of breath hold), e.g., 10 seconds. Alternatively, the LED may blink after inhalation, and continue blinking until the breath holding period has ended. In this case, the processing in the device detects the end of inhalation, turns on the LED (or causes blinking of the LED, etc.), waits the defined period of time, and then turns off the LED. Similarly, the device can emit audio indications, e.g., one or more bursts of sound (e.g., a 5 millisecond pulse of 1000 Hz), verbal instructions to hold breath, verbal countdown, music, tune, melody, etc., at the end of inhalation to cue a patient to hold their breath for the during of the sound signals. If desired, the device may also vibrate during or upon conclusion of the breath holding period.

In certain embodiments, the device provides a combination of audio and visual methods (or sound, light and vibration) described above to communicate to the user when the breath holding period has begun and when it has ended. Or during the breath holding to show progress (e.g., a visual or audio countdown).

In other aspects, the device of the disclosure may provide coaching to inhale longer, more deeply, etc. The average peak inspiratory flow during inhalation (or dosing) can be utilized to provide coaching. For example, a patient may hear a breath deeper command until they reach 90% of their average peak inspiratory flow as measured during inspiration (dosing) as stored on the device, phone or in the cloud.

In addition, an image capture device, including cameras, scanners, or other sensors without limitation, e.g. charge coupled device (CCD), may be provided to detect and measure the ejected aerosol plume. These detectors, LED, delta P transducer, CCD device, all provide controlling signals to a microprocessor or controller in the device used for monitoring, sensing, measuring and controlling the ejection of a plume of droplets and reporting patient compliance, treatment times, dosage, and patient usage history, etc., via Bluetooth, for example.

In certain embodiments, the reservoir/cartridge module may include components that may carry information read by the housing electronics including key parameters such as ejector mechanism functionality, drug identification, and information pertaining to patient dosing intervals. Some information may be added to the module at the factory, and some may be added at the pharmacy. In certain embodiments, information placed by the factory may be protected from modification by the pharmacy. The module information may be carried as a printed barcode or physical barcode encoded into the module geometry (such as light transmitting holes on a flange which are read by sensors on the housing). Information may also be carried by a programmable or non-programmable microchip on the module which communicates to the electronics in the housing.

By way of example, module programming at the factory or pharmacy may include a drug code which may be read by the device, communicated via Bluetooth to an associated user smartphone and then verified as correct for the user. In the event a user inserts an incorrect, generic, damaged, etc., module into the device, the smartphone might be prompted to lock out operation of the device, thus providing a measure of user safety and security not possible with passive inhaler devices. In other embodiments, the device electronics can restrict use to a limited time period (perhaps a day, or weeks or months) to avoid issues related to drug aging or build-up of contamination or particulates within the device housing.

The respiratory droplet delivery device may further include various sensors and detectors to facilitate device activation, spray verification, patient compliance, diagnostic mechanisms, or as part of a larger network for data storage, big data analytics and for interacting and interconnected devices used for subject care and treatment, as described further herein. Further, the housing may include an LED assembly on a surface thereof to indicate various status notifications, e.g., ON/READY, ERROR, etc.

All publications and patent applications cited in this specification are herein incorporated by reference as if each individual publication or patent application were specifically, and individually, indicated to be incorporated by reference.

While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings without departing from the essential scope thereof. Therefore, it is intended that the invention not be limited to the particular embodiment disclosed as the best mode contemplated for carrying out this invention, but that the invention will include all embodiments falling within the scope of the appended claims.

1. A droplet delivery device comprising a removable face seal that moves with a cover for a mouthpiece of the device and is configured to couple to an ejector mechanism that couples to a fluid cartridge.

2. The device of claim 1, wherein the face seal is coupled within the cover configured to removably couple to a mouthpiece with an air flow opening in fluid communication with the ejector mechanism.

3. The device of claim 2, wherein the face seal couples to an O-ring.

4. The device of claim 3, wherein the face seal is operably coupled to a spring mechanism of the cover configured to actuate the face seal.

5. The device of claim 2, wherein the face seal is operably coupled to a spring mechanism of the cover configured to actuate the face seal.

6. The device of claim 1, wherein the face seal is operably coupled to a spring mechanism of the cover configured to actuate the face seal.

7. The device of claim 6, wherein the face seal is configured to block a plurality of air flow paths within the droplet delivery device when coupled to the ejector mechanism.

8. The device of claim 1, wherein the face seal is configured to block a plurality of air flow paths within the droplet delivery device when coupled to the ejector mechanism.

9. The device of claim 2, wherein the face seal is configured to block a plurality of air flow paths within the droplet delivery device when coupled to the ejector mechanism.

10. The device of claim 3, wherein the face seal is configured to block a plurality of air flow paths within the droplet delivery device when coupled to the ejector mechanism.

11. The device of claim 4, wherein the face seal is configured to block a plurality of air flow paths within the droplet delivery device when coupled to the ejector mechanism.

12. The device of claim 5, wherein the face seal is configured to block a plurality of air flow paths within the droplet delivery device when coupled to the ejector mechanism.

13. A droplet delivery device comprising:
a body housing;
a fluid cartridge coupled to the body housing;
an ejector mechanism coupled to the fluid cartridge;
a mouthpiece with an air flow opening in fluid communication with the ejector mechanism, and wherein the mouthpiece is coupled to the body housing;
a face seal coupled to a cover configured to removably couple to the mouthpiece.

14. The device of claim 13, wherein the face seal is operably coupled to a spring mechanism of the cover configured to actuate the face seal.

15. The device of claim 14, wherein the face seal is configured to block a plurality of air flow paths within the droplet delivery device when the cover is coupled to the mouthpiece.

16. The device of claim 13, wherein the face seal is configured to block a plurality of air flow paths within the droplet delivery device when the cover is coupled to the mouthpiece.
